(19) United States
(12) Patent Application Publication
(10) Pub. No.: US 2025/0034223 A1
(43) Pub. Date: Jan. 30, 2025

(54) COMPOSITIONS AND METHODS FOR EXPANDING EX VIVO NATURAL KILLER CELLS AND THERAPEUTIC USES THEREOF

(71) Applicant: CASE WESTERN RESERVE UNIVERSITY, Cleveland, OH (US)

(72) Inventor: David N. Wald, Shaker Heights, OH (US)

(21) Appl. No.: 18/799,113

(22) Filed: Aug. 9, 2024

Related U.S. Application Data
(62) Division of application No. 16/614,132, filed on Nov. 15, 2019, now Pat. No. 12,060,577.
(60) Provisional application No. 62/508,666, filed on May 19, 2017.
(60) Provisional application No. 62/538,961, filed on Jul. 31, 2017.
(60) Provisional application No. 62/578,742, filed on Oct. 30, 2017.

(51) Int. Cl. C07K 14/54; A61K 39/00; C12N 5/0783

(52) U.S. Cl. CPC C07K 14/54; A61K 39/4613; A61K 39/4644; A61K 2239/31; A61K 2239/38; A61K 2239/48; A61K 2239/50; A61K 2239/55; C12N 5/0646; C12N 2501/2302; C12N 2501/2312; C12N 2501/2315; C12N 2501/2318; C12N 2501/2321; C12N 2502/30; C12N 2510/00

(57) ABSTRACT

The present disclosure relates to methods for expanding and increasing the cytotoxic activity of natural killer cells comprising co-culturing, as feeder cells, a population of myeloid leukemia cells engineered to express one or more of membrane-bound IL-21 (mbIL-21) or membrane-bound IL-15 (mbIL-15) in the presence of cytokine support. The present disclosure also relates to a population of acute myeloid leukemia cells engineered to express one or more of membrane-bound IL-21 (mbIL-21) or membrane-bound IL-15 (mbIL-15). The present disclosure also relates to methods of treating cancer employing the step of expanding natural killer cells using feeder cells engineered to express one or more of membrane-bound IL-21 (mbIL-21) or membrane-bound IL-15 (mbIL-15).

### RELATED APPLICATIONS

This application is a divisional of U.S. application Ser. No. 16/614,132, filed Nov. 15, 2019, which is a National Stage application of PCT international application PCT/US2018/033668, filed on May 21, 2018, which claims the benefit of U.S. Provisional Application No. 62/578,742 filed Oct. 30, 2017; U.S. Provisional Application No. 62/538,961 filed Jul. 31, 2017; and U.S. Provisional Application No. 62/508,666 filed May 19, 2017, all of which are hereby incorporated by reference in their entireties.

### REFERENCE TO A SEQUENCE LISTING

The material in the XML file, named “CASEU-57518US5-ST26.xml”, created Aug. 8, 2024, file size of 45,056 bytes, is hereby incorporated by reference.

### TECHNICAL FIELD

The disclosure relates generally to methods for expanding and enhancing the cytotoxic activity of natural killer cells to produce a large amount of highly active natural killer cells. The disclosure also relates to a genetically modified cell line for use as a feeder cell line for natural killer cell expansion. The disclosure further relates to methods of treating a variety of cancers using natural killer cells expanded by the disclosed methods.

### BACKGROUND

A number of cancers are, at present, incurable. For others, chemotherapy is only partially effective and a significant proportion of patients relapse following treatment. Some haematological malignancies can be treated by hematopoietic stem cell transplantation (HSCT), but fewer than 30% of patients requiring HSCT have a suitable donor and are the requisite age. Thus, new strategies are required.

Natural killer (NK) cells are present in an amount of about 10-15% of white blood cells in the blood of normal people, and have high killing ability when they react with non-self material. Natural killer cells non-specifically and immediately act in response to the infection of cells with various viruses, the penetration of bacteria or the production of abnormal cells to thereby remove foreign matter. They kill malignant cells without antigen-specific receptor recognition. Cancer cells commonly upregulate ligands for NK cell activating receptors such as Major histocompatibility complex (MHC) class I chain-related protein A (MICA) and MHC class I chain-related protein (MICB) and downregulate ligands for inhibitory receptors such as HLA class I. In particular, cancer cells routinely downregulate HLA to avoid T-cell detection, making these cells paradoxically sensitive to NK cell killing. As normal cells express HLA class I (as well as lower levels of activating ligands), they are protected from NK cell-mediated killing. While T cell therapy, particularly chimeric antigen receptor therapy (CAR-T) has shown enormous promise for B cell malignancies, this strategy typically requires individual donor cells to be clinically manufactured for each patient, making it an extremely expensive process. These cells have also only shown significant clinical efficacy for a subset of patients with lymphoid leukemia to date. In addition, while allogeneic T cells cause severe graft-vs-host disease (GVHD), allogeneic NK cells have been found to be safe and do not cause GVHD in patients.

To date, three key obstacles have impeded the clinical use of NK cells, including: 1) lack of a robust ex vivo NK cell expansion system; 2) insufficient NK cell activity against tumor cells; and 3) maintenance and proliferation of NK cells in cancer patients despite a highly suppressive tumor microenvironment. For these reasons, methods for the proliferation and production of effective natural killer cells are required. Provided herein is a genetically manipulated feeder cell line (termed “NKF”) that provides robust expansion of NK cells ex vivo. Also provided herein are methods for expanding and activating NK cells employing NKF cells. Further provided herein are methods of treating a cancer with NK cells produced by the novel expansion platform. In certain embodiments, the NK cells can be provided by a universal donor, abrogating the need for individualized therapy approaches.

### SUMMARY

According to the present disclosure, in a first aspect, is a composition comprising acute myeloid leukemia (AML) cells engineered to express a membrane-bound interleukin (mbIL) protein.

In an example of the first aspect, the mbIL protein comprises a polypeptide sequence of SEQ ID NO:1, SEQ ID NO:3, sequences having greater than 80% sequence homology with SEQ ID NO:1, sequences having greater than 80% sequence homology with SEQ ID NO:3, or combinations thereof.

In another aspect of the first aspect, the mbIL protein comprises a polypeptide sequence of SEQ ID NO:1 or sequences having greater than 80% sequence homology with SEQ ID NO:1.

In yet another example of the first aspect, the mbIL protein comprises a polypeptide sequence of SEQ ID NO:3 or sequences having greater than 80% sequence homology with SEQ ID NO:3.

In another example of the first aspect, the mbIL protein comprises a polypeptide sequence encoded by the nucleotide sequence of SEQ ID NO: 2 or SEQ ID NO:4.

In another example of the first aspect, the nucleotide sequence of SEQ ID NO:2 encodes the polypeptide of SEQ ID NO:1.

In yet another example of the first aspect, the nucleotide sequence of SEQ ID NO:4 encodes the polypeptide of SEQ ID NO:2.

In another example of the first aspect, the AML cells are OCI-AML3 cells.

In yet another example of the first aspect, the cells are HL-60 cells.

In a second aspect, there is a method for expanding natural killer (NK) cells ex vivo, said method comprising harvesting donor cells from a donor subject; isolating the NK cells; and expanding the NK cells in the presence of OCI-AML3 feeder cells; or OCI-AML3 feeder cells and one or more of IL-2, IL-15, ALT-803, hetIL-15, IL-12, IL-18, IL-21 or derivatives or fragments thereof.

In an example of the second aspect, the OCI-AML3 feeder cells are engineered express to a membrane-bound interleukin (mbIL) protein.

In another example of the second aspect, the mbIL protein comprises a polypeptide sequence of SEQ ID NO:1, SEQ ID NO:3, sequences having greater than 80% sequence homology with SEQ ID NO:1, sequences having greater than 80% sequence homology with SEQ ID NO:3, or combinations thereof.

In an example of the second aspect, the NK cells are genetically modified to express one or proteins capable of providing cytokine support. In certain exemplary aspects, the proteins capable of providing cytokine support are selected from the group consisting of mbIL-15, soluble IL-15, soluble IL-21, mbIL-21, mbIL-2, or soluble IL-2.

In another aspect of the second embodiment, the NK cells are genetically modified to express one or proteins capable of inhibiting TGFβ signaling. In one exemplary aspect, the proteins capable of inhibiting TGFβ signaling is a dominant negative TGFβRII in which there is a truncation of the cytoplasmic domain.

In another example of the second aspect, the donor cells are autologous.

In another example of the second aspect, the donor cells are allogenic.

In yet another example of the second aspect, the donor cells are harvested from a living donor.

In another example of the second aspect, the donor cells are harvested from a cord blood.

In yet another example of the second aspect, a ratio of feeder cells:NK cells is at least 1:1 based on an amount of NK cells present at the time of refreshing.

In another example of the second aspect, the ratio of feeder cells:NK cells is about 5:1.

In yet another example of the second aspect, the ratio of feeder cells:NK cells is about 10:1.

In another example of the second aspect, the step of expanding the NK cells provides at least a 10,000-fold expansion within 4 weeks.

In another example of the second aspect, the step of expanding the NK cells provides at least a 20,000-fold expansion.

In yet another example of the second aspect, the step of expanding the NK cells provides at least a 30,000-fold expansion.

In another example of the second aspect, the step of expanding the NK cells continues for at least 4 weeks.

In another example of the second aspect, the step of expanding the NK cells continues for at least 6 weeks.

In yet another example of the second aspect, the feeder cells are refreshed weekly throughout the step of expanding the NK cells.

In a third aspect, there is a method of treating a recipient subject in need thereof, said method comprising: harvesting donor cells from a donor subject; isolating the natural killer (NK) cells; expanding the NK cells in the presence of OCI-AML3 feeder cells; or OCI-AML3 feeder cells and one or more of IL-2, IL-15, ALT-803, hetIL-15, IL-12, IL-18, IL-21 or derivatives or fragments thereof; and administering the expanded cells to the recipient subject.

In an example of the third aspect, the OCI-AML3 feeder cells are engineered express to a membrane-bound interleukin (mbIL) protein.

In another example of the third aspect, the mbIL protein comprises a polypeptide sequence of SEQ ID NO:1, SEQ ID NO:3, sequences having greater than 90% sequence homology with SEQ ID NO:1, sequences having greater than 90% sequence homology with SEQ ID NO:3, or combinations thereof.

In another example of the third embodiment, the NK cells are genetically modified to express one or proteins capable of providing cytokine support. In certain exemplary aspects, the proteins capable of providing cytokine support are selected from the group consisting of mbIL-15, soluble IL-15, soluble IL-21, mbIL-21, mbIL-2, or soluble IL-2.

In another aspect of the third embodiment, the NK cells are genetically modified to express one or more proteins capable of inhibiting TGFβ signaling. In one exemplary aspect, the proteins capable of inhibiting TGFβ signaling is dominant negative TGF-beta RII in where there is a truncation of the cytoplasmic domain.

In an example of the third aspect, the donor subject and the recipient subject are the same human.

In another example of the third aspect, the donor subject and the recipient subject are different humans.

In another example of the third aspect, the donor cells are harvested from cord blood.

In yet another example of the third aspect, the expanded cells are administered as monotherapy.

In another example of the third aspect, the expanded cells are administered concomitantly or sequentially with at least one additional therapeutic.

In another example of the third aspect, the at least one additional therapeutic is a cytokine support administered concomitantly.

In another example of the third aspect, the cytokine support is selected from the group consisting of IL-15, IL-2, ALT-803, IL-18, IL-12, IL-21 and derivatives and combinations thereof.

In another example of the third aspect, the additional therapeutic is IL-15 or a derivative thereof.

In yet another example of the third aspect, the at least one additional therapeutic is an inhibitor of an immunoregulatory protein.

In another example of the third aspect, the inhibitor of an immunoregulatory protein is selected from the group consisting of a TGF-beta inhibitor, a GSK3 inhibitor, a PD1 inhibitor, a PDL1 inhibitor, a LAG-3 inhibitor, a TEVI-3 inhibitor, a TIGIT inhibitor, and combinations and/or derivatives thereof.

In another example of the third aspect, the inhibitor of an immunoregulatory protein is a TGF-beta inhibitor.

In another example of the third aspect, the TGF-beta inhibitor is selected from the group consisting of galunisertib, EW7203, EW7197, belagepumatucel-L, fresolimumab, gemogenovatucel-T, trabedersen, XOMA089, and a genetic modification of the cells with a dominant negative TGF-beta RII.

In another example of the third aspect, wherein the expanded cells are administered concomitantly with a cytokine support and a TGF-beta inhibitor.

In yet another example of the third aspect, the expanded cells are administered in a dose of at least 10⁶ cells/kg.

In another example of the third aspect, the expanded cells are administered in a dose of at least 10⁹ cells/kg.

In an example of the third aspect, the recipient in need thereof has a cancer.

In certain examples of the third aspect, the cancer is a solid tumor, a blood cancer or a metastatic cancer.

In yet another example of the third aspect, the blood cancer is selected from the group consisting of AML, myeloma, T cell leukemia, Non-Hodgkin's Lymphoma and B cell leukemia, Myelodysplastic syndromes, CLL, and CML.

In another example of the third aspect, the solid tumor is selected from the group consisting of a colon cancer, a prostate cancer, a sarcoma, a pancreatic cancer, and a breast cancer.

In another example of the third aspect, the cancer is a metastatic cancer.

In yet another example of the third aspect, the metastatic cancer is one or more of a lung metastasis or a liver metastasis.

In another example of the third embodiment, the recipient in need thereof has a viral infection. In some aspects, the viral infection is coxsackievirus, human immunodeficiency virus (HIV), hepatitis C virus (HCV), influenza virus, poxvirus, or herpesviruses.

Additional features and advantages will be set forth in the detailed description which follows, and in part will be readily apparent to those skilled in the art from that description or recognized by practicing the embodiments as described herein, including the detailed description which follows, the claims, as well as the appended drawings.

It is to be understood that both the foregoing general description and the following detailed description are merely exemplary, and are intended to provide an overview or framework to understanding the nature and character of the claims. The accompanying drawings are included to provide a further understanding, and are incorporated in and constitute a part of this specification. The drawings illustrate one or more embodiments, and together with the description serve to explain principles and operation of the various embodiments. Directional terms as used herein—for example, up, down, right, left, front, back, top, bottom—are made only with reference to the figures as drawn and are not intended to imply absolute orientation.

### BRIEF DESCRIPTION OF THE DRAWINGS

The above and other features, examples and advantages of aspects or examples of the present disclosure are better understood when the following detailed description is read with reference to the accompanying drawings, in which:

FIG. 1 is a schematic representation of NKF-based NK cell expansion.

FIG. 2 is a graph demonstrating fold expansion of NK cells after 1 week incubation using HL-60 (left) and OCI-AML3 (right) cells as feeder cells provided at 5:1 ratios.

FIG. 3 is a graph demonstrating expression of mbIL-21 as determined by rt-PCR in OCI-AML3 (left) and OCI-AML3-mbIL-21 (right) cells.

FIG. 4a is a graph demonstrating fold expansion of NK cells expanded with varying NKF:NK cell ratios.

FIG. 4b is a graph demonstrating fold expansion of NK cells expanded with varying concentrations of IL-2.

FIG. 5 is a graph demonstrating exemplary proliferation of NK cells expanded according to the disclosed methodology.

FIG. 6a presents representative flow plots demonstrating purity of NK cells in the initial PBMC cell mixture (bottom row) and after expansion (top row).

FIG. 6b is a graph demonstrating relative fractions of cell types in NKF-expanded NK cells.

FIG. 7 is a graph demonstrating the high in vitro activity of NKF-expanded NK cells against a variety of cancer cell lines as compared to IL-2 activated non-expanded cells.

FIG. 8 is a graph demonstrating the high in vitro activity of NKF-expanded NK cells against a variety of cancer cell lines provided at varying NKF:NK cell ratios.

FIG. 9 is a graph demonstrating that NKF-expanded NK cells were efficiently trafficked to the liver and lungs.

FIG. 10a is a graph demonstrating the enhancement in cell cytotoxicity of ALT-803 compared to IL-2 as cytokine support in vitro.

FIG. 10b depicts in vivo cytotoxicity of NKF-expanded NK cells against OCI-AML3 cells in NSG mice.

FIG. 10c depicts subcutaneous tumor volume of control and NKF-expanded NK cells in the presence of IL-2 or ALT-803 as cytokine support.

FIG. 10d provides quantification of lung tumors following treatment with control and NKF-expanded NK cells in the presence of IL-2 or ALT-803.

FIG. 10e shows Ki67 lung staining of lung metastases.

FIG. 10f compares the enhanced lung trafficking of NK cells provided with ALT-803 to NK cells provided with IL-2.

FIG. 11 is a graph showing the decreased in vitro cytotoxicity of NK cells in the presence of TGF-beta and the rescue of NK cell efficacy with co-administration of a TGF-beta inhibitor.

FIG. 12a shows exemplary tumor sections showing colon cancer metastases to the liver following treatment with vehicle, NK cells alone, a TGF-beta inhibitor alone or NK cells in combination with a TGF-beta inhibitor.

FIG. 12b shows the quantification of the liver sections of FIG. 12b, provided as the ratio of % tumor area:total tissue area.

### DETAILED DESCRIPTION

Example embodiments will now be described more fully hereinafter with reference to the accompanying figures in which example embodiments and representative data are shown. Whenever possible, the same reference numerals are used throughout the drawings to refer to the same or like parts. However, the embodiments may take on many different forms and should not be construed as limited to those specifically set forth herein. These example embodiments are provided so that this disclosure will be both thorough and complete, and will fully convey the scope of the claims to those skilled in the art.

Directional terms as used herein (e.g., up, down, right left, front, back, top, bottom) are made only with reference to the figures as drawn and are not intended to imply absolute orientation.

As used herein, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. When the term “about” is used in describing a value or an end-point of a range, the disclosure should be understood to include the specific value or end-point referred to. Whether or not a numerical value or end-point of a range in the specification recites “about,” the numerical value or end-point of a range is intended to include two embodiments: one modified by “about,” and one not modified by “about.” It will be further understood that the endpoints of each of the ranges are significant both in relation to the other endpoint, and independently of the other endpoint.

The terms “substantial,” “substantially,” and variations thereof as used herein are intended to note that a described feature is equal or approximately equal to a value or description. For example, a “substantially planar” surface is intended to denote a surface that is planar or approximately planar. Moreover, “substantially” is intended to denote that two values are equal or approximately equal. In some embodiments, “substantially” may denote values within about 10% of each other, such as within about 5% of each other, or within about 2% of each other.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. Thus, cells that are “free of” or “substantially free of T cell contamination” for example, are cells to which T cells are not actively added or batched into cell culture, but may be present in very small as a contaminant resulting from natural cell progression during expansion. Similarly, other components may be characterized as “free of” or “substantially free of” in the same manner.

Further, as used herein, the term “consisting essentially of” allows for elements not explicitly recited, but excludes element that affect basic or novel characteristics of the inventions.

Natural killer (NK) cells are potent effectors of the innate immune system with cytotoxic and immunoregulatory function. Human NK cells are typically characterized as lymphocytes expressing CD56 or CD16 and lacking CD3 expression, and are estimated to comprise up to about one-third of peripheral blood lymphocytes in normal subjects. Unlike T-cells, NK cells recognize targets in a major histocompatibility complex (MHC)-unrestricted manner. NK cells play an important role in initiating responses to infection, engraftment, graft-versus-host disease, anti-leukemic activity and post-transplant infection, some of which are reviewed in Lowdell, M. W. Natural killer cells in haematopoietic stem cell transplantation. Transfusion Medicine 2003; 13:399-404.

Regarding cancer specifically, because cancer cells often have reduced or no class I MHC expression, they can become targets of NK cells, and thus, adoptive immunotherapy with NK cells has emerged as a promising anti-cancer treatment. NK cells have therapeutic potential for a wide variety of human malignancies, including sarcomas, myeloma, carcinomas, lymphomas, and leukemias. Until recently, the clinical efficacy and effective application of NK cell immunotherapy has been limited by the inability to obtain sufficient cell numbers for adoptive transfer, as these cells represent a small fraction of peripheral white blood cells, expand poorly ex vivo, and have limited life spans in vivo. The ability to harvest large numbers of peripheral blood lymphocytes through leukapheresis, deplete alloreactive T cells, and activate the remaining NK cells with IL-2 has enabled NK cell adoptive immunotherapy, but this process is expensive, invasive, and remains limited in cell dose to a single infusion of typically less than 2×10⁷ NK cells/kg.

There are various known methods for isolating NK cells from peripheral blood. Generally, to isolate natural killer cells from peripheral blood, PBMCs are separated into lymphocytes and monocytes, and the lymphocytes are further divided into T cells, B cells, and natural killer cells for isolation.

In the present disclosure, the “peripheral blood mononuclear cells,” “PBMCs” or “mononuclear cells” refer to mononuclear cells separated from peripheral blood typically used for anti-cancer immunotherapy. The peripheral blood mononuclear cells can be obtained from human blood collected using known methods such as the Ficoll-Hypaque density gradient method. PMBCs may be obtained from a healthy person, a patient at risk of cancer, or a cancer patient. The PBMCs used herein can be, but do not necessarily need to be, autologous; allogeneic PBMCs may also be used to induce and proliferate the NK cells for anti-cancer immunotherapy according to the present disclosure.

In some embodiments, NK cells may be derived from a subject and grown in vitro to provide a population of NK cells for use in the present disclosure. In deriving NK cells from a subject, the cells may come from stem cells or they may be collected from a living donor. In a preferred aspect, the NK cells employed herein are collected from a living donor. In certain aspects, the living donor many be a human living donor. In an alternative embodiment, a NK cell known in the art that has previously been isolated and cultured may be used in the present invention. Thus an established NK cell line may be used. Many such NK cells lines are commercially available and known to those in the art.

Once isolated, NK cells must be expanded if larger numbers are desired. As used herein, “expanded” refers to the increase in number of NK cells by any method. Though several expansion platforms have been developed for NK cells, few have the potential to efficiently produce a large magnitude of highly active NK cells. Such prior art exemplary systems include:
1) Cytokine cocktails: These systems employ various combinations of cytokines (e.g. IL-15/IL-21) and typically lead to several dozen fold expansion. (Wagner J, at al. A Two-Phase Expansion Protocol Combining Interleukin (IL)-15 and IL-21 Improves Natural Killer Cell Proliferation and Cytotoxicity against Rhabdomyosarcoma. Frontiers in immunology. 2017; 8:676.)
2) EBV-LCL feeder line: This system involves an EBV infected feeder line and therefore only a single feeder cell stimulation can be added to the NK cells to ensure eradication of the EBV infected cells which significantly limits the expansion potential of the NK cells. (Berg M, et al. Clinical-grade ex vivo-expanded human natural killer cells upregulate activating receptors and death receptor ligands and have enhanced cytolytic activity against tumor cells. Cytotherapy. 2009; 11(3):341-55.)
3) NK-92 cells: This is an NK cell leukemia cell line that has been used clinically and commercialized by at least two companies. However, these cells must be irradiated as they are cancer cells and that prevents their proliferation in vivo. These cells also have a very short persistence in vivo (˜48 hr). (Tonn T, et al. Treatment of patients with advanced cancer with the natural killer cell line NK-92. Cytotherapy. 2013; 15(12):1563-70.)
4) K562 mIL-21 feeder line: This feeder line is often considered the gold standard platform and leads to very similar proliferation and NK cell activation as the NKF cell platform disclosed herein. Despite the promise of these feeder cells, legal issues with multiple institutions have prevented their further development and these cells are no longer being commercialized and cannot be used for future clinical studies. (Denman C J, et al. Membrane-bound IL-21 promotes sustained ex vivo proliferation of human natural killer cells. PloS one. 2012; 7(1):e30264.)
5) Cord blood derived NK cells: The major limitation of using cord blood is a very low starting NK cell population, which limits the scale of proliferation and has significantly higher costs (purchase of cord blood units, differentiation growth factors etc.) (Spanholtz J, et al. High log-scale expansion of functional human natural killer cells from umbilical cord blood CD34-positive cells for adoptive cancer immunotherapy. PloS one. 2010; 5(2):e9221.)

One of the advantages of the systems and methods disclosed herein is the ability to efficiently produce a large magnitude of highly active NK cells. In contrast to most of the prior art expansion systems, including those mentioned above, in certain embodiments of the present disclosure the step of expanding the NK cells provides at least a 10,000-fold expansion within four weeks, such as at least 12,000-fold, at least 14,000-fold, least 16,000-fold, least 18,000-fold, least 20,000-fold, least 22,000-fold, least 24,000-fold, least 26,000-fold, least 28,000-fold, at least 30,000-fold, or at least 32,000-fold expansion within four weeks. In other words, a 4 to 5 week NK cell expansion starting with a peripheral blood pheresis sample from a normal donor (approximately 350×10⁶ NK cells) will yield approximately 10¹³ NK cells. Since high dose NK cell infusions are typically around 10⁹/kg, this could yield up to 100 doses per single expansion.

In some aspects of the present disclosure, the administration of the NK cell may be non-immunogenic, for example, by providing a conditioning regimen (e.g. cyclophosphamide and fludarabine) to the patient at the time of administration. The term “non-immunogenic” is thus used broadly herein to mean that when the cell is injected into or otherwise administered to a subject, it avoids detection by the body's immunological system and is not rejected or recognized as foreign. More particularly, the cell does not raise (or is not capable of raising) an immune response sufficient to lead to rejection of the cell and/or to affect the function of the cells. Thus, the cells retain cytotoxic activity in the subject, more particularly, significant or substantial or measurable cytotoxic activity against a target cell. As with any biological system, the absence of an immune response may not be absolute (or 100%), A small (or mild or minor) immune response to the NK cell (e.g. a de minimis immune response) may be tolerated, as long as the function or utility of the cells is not substantially affected (i.e. as long as the cells can still perform their function). That is, the NK cells employed herein may be “universal” in nature such that one set of donor cells can be used for virtually any patient without generating a negative immune response.

Such a universal donor approach will allow for “off-the-shelf” NK cells that will dramatically reduce the cost and increase the accessibility of the disclosed therapeutic strategy worldwide. The ability to harvest allogeneic NK cells from one donor and expand those cells to obtain the doses needed for dozens of unrelated patients would address the major limits of this therapeutic approach. NK cells, unlike T-cells, does not cause GVHD, and therefore, it is thought that they can be safely administered without regard to HLA matching. Besides cost and feasibility, another benefit of the universal donor approach is the potential for identifying donors that are predicted to have the most cytotoxic NK cells. Stem cell transplant donors with a genotype that encodes a preponderance of activating KIRs (B-haplotypes) have been shown to result in improved clinical outcomes (disease free survival, overall survival and GVHD) following allogeneic stem cell transplantation for myeloid leukemia. Finally, utilizing a donor not matched by HLA to recipients also increases the potential for NK cell alloreactivity to promote a “graft vs. leukemia” effect, which has been found to improve disease free survival in certain cancer populations.

In this regard, the NK cells of the present invention may be autologous or allogeneic NK cell. “Autologous” NK cells are cells derived from the patient. “Allogeneic” NK cells are derived from another individual, having non-identical gene at one or more loci. If the NK cells are derived from an identical twin, they may be termed “syngeneic”. In particularly preferred embodiments, the NK cells employed according to the disclosed methods, including the methods of treating cancer, are allogeneic NK cells.

To address the limitations on widespread use of NK cell adoptive transfer therapy, the disclosure herein provides a composition of feeder cells methods employing such which allow for the robust expansion of an NK cell system. As used herein, the term “feeder cells” refers to cells that, due to their metabolic activity, produce various metabolites to thereby assist in the proliferation of target cells, even though these cells cannot themselves proliferate.

Feeder cells as used according to the present disclosure may be any population of leukemia cells engineered to express a membrane-bound interleukin (mbIL). As used herein, the term “interleukin (IL) protein” refers to a collection of biologically active cytokines produced by immune cells such as lymphocytes, monocytes or macrophages. According to the present disclosure, the term “cytokine” refers to an immune activating cytokine (secreted protein and/or signaling molecule) that can be used to induce NK cells isolated from PBMCs. Examples of IL proteins which may be used in the present disclosure include IL-2, IL-7, IL-12, IL-15, IL-18, IL-21, Flt3-L, SCF, IL-7 and the like.

In certain aspects of the disclosure, the feeder cells are HL-60 cells or OCI-AML3 cells. In a preferred aspect, the feeder cells are OCI-AML3 cells. Preferably, the mbIL comprises one or more of IL-15 or IL-21. In a preferred aspect, the mbIL consists of, or consists essentially of, mbIL-15. In another preferred aspect, the mbIL consists of, or consists essentially of, mbIL-21.

The cells that are used as feeder cells according to the present disclosure may be non-inactivated or inactivated cells whose proliferation was inhibited prior to use. More specifically, the feeder may be inactivated to ensure their safety and to eliminate their potential to proliferate when employed as part of the feeding platform described herein. A common method for inactivating feeder cells comprises the step of irradiating the killer cells with gamma-rays. If non-inactivated feeder cells are used, they can be killed by natural killer cells during culture because they are tumor cells. In a preferred aspect of the present disclosure, the feeder cells are inactivated using gamma radiation prior to adding them to the cell culture comprising NK cells. In some embodiments, the feeder cells can be inactivated using 10 Gy or greater or of radiation, such as 10 Gy, 20 Gy, 30 Gy, 40 Gy, 50 Gy, 60 Gy, 70 Gy, 80 Gy, 90 Gy, 100 Gy, 110 Gy, 120, Gy, 130 Gy, 140 Gy, 150 Gy, 175 Gy, 200 Gy, 225 Gy, 250 Gy, 300 Gy, 350 Gy, 400 Gy, 450, Gy or 500 Gy. In preferred methods, the feeder cells are inactivated using 90 Gy of gamma radiation.

As used herein, the term “cytotoxic” refers to a cell capable of inducing cell death by lysis or apoptosis in a target cell.

The term “target cell” refers to any cell which is killed by the cytotoxic cells of the invention. More particularly, the target cell is the cell targeted by the NK cells. Thus the target cell may be any cell in the body of the subject which it is desired to abrogate, e.g. to remove, kill, render inactive etc. Preferred target cells are virally infected cells and cancer cells, but target cells may be any cells resulting from a disease or clinical condition that may benefit from adoptive cell transfer therapy. As well as cancer cells, other cells that it may be clinically desirable to remove include infected cells, that is cells infected with any pathogen such as virus-infected cells, or cells infected with any other pathogenic organism, e.g. any microorganism, for example, bacteria, fungi, mycoplasma, protozoa, or prions.

Due to their high cytotoxicity against cancer cells, the adoptive transfer of NK cells into cancer patients is a promising therapeutic strategy. As used herein, the term “adoptive transfer” means the transfer of cells into a patient. Support for NK cells use in cancer therapy can be seen from the correlation of the presence and activity of tumor-infiltrating NK cells in a variety of cancers. Several types of cancers, including hematologic malignancies, sarcoma, neuroblastoma, and ovarian cancer have been found to be particularly sensitive to NK cell therapy, and in some cases, improved outcomes have been observed. However, three key obstacles have impeded the widespread clinical use of NK cells as set forth above, including: 1) lack of a robust ex vivo NK cells expansion system; 2) insufficient NK cell activity; and 3) maintenance of activity and proliferation of NK cells in cancer patients despite a highly immunosuppressive tumor microenvironment.

To address the limitations of lack of a robust expansion platform and insufficient NK cell activity, the present disclosure reports a novel ex vivo expansion platform (termed “NKF”) that leads to vastly improved numbers of highly active NK cells. The NKF platform described herein employs a feeder cell line constructed from an AML cells line transduced with a membrane-bound IL protein. In some aspects of the present disclosure, OCI-AML3 cells, HL-60 cells, or combinations thereof, are employed as the feeder cell line. Preferably, OCI-AML3 cells are employed as the feeder cell line.

Without wishing to be bound by theory, the presence of membrane-bound IL proteins such as membrane-bound IL-15 (mbIL-15) or membrane-bound IL-21 (mbIL-21) is believed to prevent NK cells from undergoing senescence, markedly improving their ability to expand ex vivo. There are a myriad of studies proposing how and why cytokines exert their effect on NK cell function and prevention of senescence, but a single, clear explanation has not yet emerged. The feeder cells of the present disclosure preferably comprise mbIL-15, mb-IL-21 or combinations thereof as the membrane-bound IL protein.

The polypeptide sequence of mbIL-21 as employed in the present disclosure is identified in SEQ ID NO:1. In certain aspects of the present disclosure, the feeder cell line has been engineered to express a polypeptide of SEQ ID NO:1, or a sequence having at least 80% sequence identity (or sequence homology) to the amino acid sequence of SEQ ID NO:1, such as 80%, 85%, 90%, 95%, 99% or 100% sequence homology to SEQ ID NO:1. Alternatively, the feeder cells can be modified by introducing a polynucleotide that encodes a polypeptide having at least 80% sequence identity to the amino acid sequence of SEQ ID NO:1. Once such polynucleotide includes SEQ ID NO:2.

The polypeptide sequence of mbIL-15 as employed in the present disclosure is identified in SEQ ID NO:3. In certain aspects of the present disclosure, the feeder cell line has been engineered to express a polypeptide of SEQ ID NO:3, or a sequence having at least 80% sequence identity to the amino acid sequence of SEQ ID NO:3, such as 80%, 85%, 90%, 95%, 99% or 100% sequence homology to SEQ ID NO:3. Alternatively, the feeder cells can be modified by introducing a polynucleotide that encodes a polypeptide having at least 80% sequence identity to the amino acid sequence of SEQ ID NO:3. Once such polynucleotide includes SEQ ID NO:4.

In certain aspects of the present disclosure, the feeder cells have been engineered to express both mbIL-15 and mbIL-21. In other words, the feeder cells have been engineered to express both a polypeptide of SEQ ID NO:1, or a sequence having at least 80% sequence identity to the amino acid sequence of SEQ ID NO:1 and a polypeptide of SEQ ID NO:3, or a sequence having at least 80% sequence identity to the amino acid sequence of SEQ ID NO:3.

The feeder cells according to the present disclosure can be further modified to express one or more associated accessory signaling polypeptides, cytokines or fragments thereof. Such expression may correlate with increased expression of the mbIL proteins in certain aspects.

“Sequence homology” or “sequence identity” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

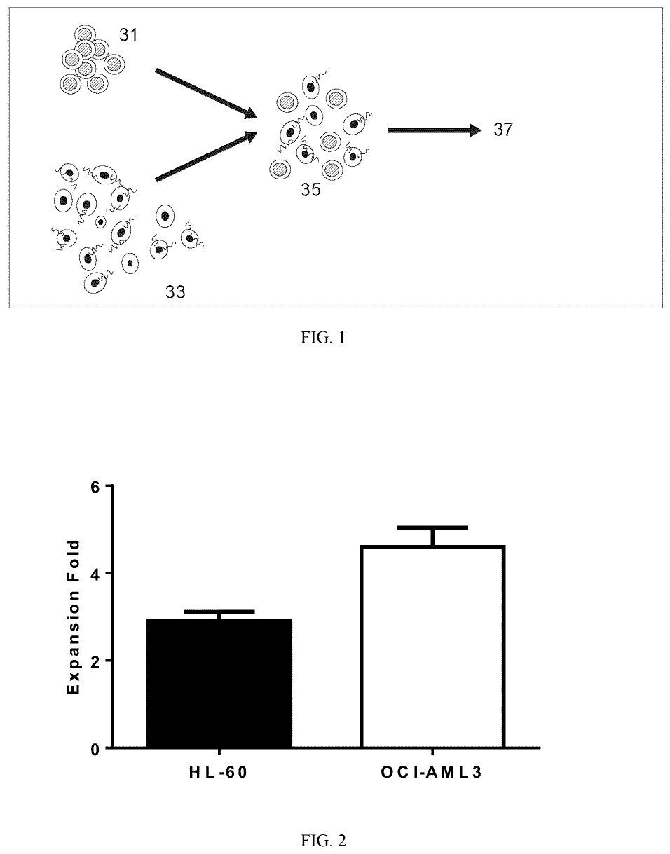

Also included in the present disclosure are methods for expanding NK cells ex vivo. Referring to FIG. 1, the method comprises the steps of harvesting donor cells from a subject; isolating the NK cells 31; and expanding the NK cells in the presence of NKF (feeder) cells 33. In certain aspects, the NK cells can be co-cultured with a cytokine support 35. In other aspects of the disclosed methods, the NKF cells can be refreshed 37.

More specifically, donor cells can be harvested from a subject according to known methods, such as those detailed above. As noted above, donor cells can be cells donated from a living donor or cells harvested from cord blood. The cells can be from a human. In a preferred aspect, the cells can be harvested from a universal living human donor. Once collected, the NK cells can be isolated from PBMCs according to known methods, such as those described above, to provide isolated NK cells 31.

The isolated NK cells 31 are then expanded in the presence of NKF feeder cells 33 in a pre-determined ratio. In certain aspects, the NKF cells 33 are OCI-AML3 cells or HL-60 cells. In a preferred aspect, the feeder cell line comprises OCI-AML3 cells that have been engineered to expressed a mbIL protein, such as those detailed above. In a particularly preferred embodiment, the NKF cells are OCI-AML3 cells that have been engineered to express one or more of mbIL-15 or mbIL-21. In some aspects of the present method, the ratio of NKF cells to NK cells is preferably greater than or equal to about 1:1, such as about 1:1, about 1.5:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, or about 10:1, based on the number of NK cells counted on the day of the NKF cell addition. In some aspects, a 5:1 ratio of NKF:NK cells is particularly preferred. In other aspects of the present method, the NKF:NK cell ratio is about 10:1, or greater, such as 10:1, 15:1, 20:1, 25:1 or 30:1, based on the NK cell count on the day of the NKF cell addition. In a particularly preferred embodiment, the NKF:NK cell ratio is 10:1.

In addition to the NKF cells, NK cells can be expanded in vivo or ex vivo in the presence of cytokine support 35. Cytokine support 35 is used to enable the cells to survive and proliferate after infusion into the patient. Exemplary cytokines for use according to the disclosed methods include IL-2, IL-15, ALT-803, hetIL-15, IL-12, IL-18, IL-21 or fragments or derivatives thereof. The present methods may comprise the use of more than one cytokine support. In preferred aspects of the present methods, NK cells may be expanded in the presence of IL-2, IL-15 or ALT-803 or other IL-15 derivatives. Alternatively, cytokine support can be provided by engineering the NK cells to express additional cytokines. For example, this can be accomplished by transducing genes for one or more of mbIL-15, soluble IL-15, soluble IL-21, mbIL-21, mbIL-2, or soluble IL-2 into the NK cells prior to or after NK cell expansion. In some aspects of the disclosure, cytokine support is provided by pharmacologic inhibitors. In some aspects of the disclosure, cytokine support is provided by genetic modification. In other aspects, cytokine support can be provided both pharmacologically and genetically.

A novel IL-15 superantagonist called ALT-803 has recently been developed that offers the potential to markedly improve the survival/proliferation of adoptively infused NK cells. (Zhu, X. et al. Novel Human Interleukin-15 Agonists. J. Immunol. 2009; 183(6):3598-3607, herein incorporated by reference). ALT-803 is a fusion protein containing a mutated IL-15 molecule fused to a portion of the IL-15 receptor (αSu/Fc fusion protein). ALT-803 exhibits greater than 25-fold enhancement in biological activity compared to IL-15 and a markedly improved half-life (25 hrs).

In certain aspects of the disclosed methods, the expanding of NK cells in the presence of NKF cells can last up from one to eight weeks. That is, the step of expanding NK cells can take, e.g., one week, two weeks, three weeks, four weeks, five weeks, six weeks, seven weeks, or eight weeks.

When NK cells are expanded for more than one week, the NKF cells may need refreshed or replenished throughout the step of expanding the NK cells. Refreshing of NKF cells can be done on an as-needed basis, preferably weekly. The amount of NKF cells for refreshment can employ the same ratio of NKF:NK cells as the starting ratio or the NKF cells can be replenished in a different ratio as needed. In some aspects of the present method, the ratio of NKF cells to NK cells of refreshment is preferably greater than or equal to about 1:1, such as about 1:1, about 1.5:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, or about 10:1, based on the number of NK cells counted on the day of the NKF cell addition. In some aspects, a 5:1 ratio of NKF:NK cells is particularly preferred. In other aspects of the present method, the NKF:NK cell ratio is about 10:1, or greater, such as 10:1, 15:1, 20:1, 25:1 or 30:1, based on the NK cell count on the day of the NKF cell addition. In a particularly preferred embodiment, the NKF:NK cell ratio is 10:1.

As demonstrated below, the expansion potential of the disclosed methodology is vastly improved compared to prior art systems. In certain aspects of the claimed methods, the step of expanding the NK cells provides at least a 10,000-fold expansion of NK cells within 4 weeks. That is, after four weeks of culturing NK cells in the presence of NKF cells (replenished weekly), there are at least 10,000 more NK cells present than were present in the starting culture. In some embodiments, the step of expanding the NK cells provides at least a 20,000-fold expansion of NK cells within 4 weeks. In other embodiments of the disclosed methods, the step of expanding the NK cells provides at least a 30,000-fold expansion of NK cells within 4 weeks.

Once large numbers of NK cells can be obtained, NK cells can be used as cytotoxic agents against cancer cells. In some aspects are methods of treating cancer in a recipient subject in need thereof, said method comprising: (a) harvesting donor cells from a donor subject; (b) isolating the NK cells from the donor cells; (c) expanding the NK cells according to any of the methods detailed herein; (d) administering the expanded NK cells to the recipient subject.

As demonstrated below, NK cells expanded according to the disclosed methods demonstrated markedly increased cytotoxic activity against a wide variety of cancer cells lines, both in vitro and in vivo.

NK cell therapy has shown clinical promise against a wide variety of tumor types, including both solid tumors and blood cancers. The therapeutic approaches described herein can be used as treatment of virtually all types of cancers and pre-cancers (e.g. Myelodysplastic syndrome), including but not limited to carcinomas, sarcomas, melanomas, lymphomas, and leukemias, and having places of origin including but not limited to colon, prostate, brain, breast, liver, lung, pancreatic, bone, ovarian, skin, pancreatic, blood and others. The methods disclosed herein are contemplated for treatment of both metastatic cancers as well as primary tumor sites.

NK cell therapy has also been employed to treat viral infections, including coxsackievirus, human immunodeficiency virus (HIV), hepatitis C virus (HCV), influenza virus, poxvirus, and herpesviruses, with highly positive clinical outcomes. Without wishing to be bound by theory, it is believed that NK cells control viral infections, in part, by secreting IFNγ and TNFα. The methods disclosed herein are contemplated for treatment of viral infections. In a preferred embodiment, the methods disclosed herein can be used to treat HIV infections.

In certain aspects of the claimed methods, NK cells expanded according to the disclosed methodologies can be administered in dosages amounts ranging from 10-10¹² cells/kg. Preferred dosage amounts range from 10×10⁶ cells/kg to 10,000×10⁶ cells/kg and are preferably in the range of 1,000×10⁶ cells/kg. Dosage amounts can be adjusted on a per patient or a per administration basis as needed throughout the treatment cycle.

NK cells expanded according to the disclosed methodologies can be administered to patients via infusion. Preferably, cell transfers occur biweekly, once weekly, once every two weeks, once every three weeks, once every four weeks, once every five weeks, once every six weeks, or on an as-needed basis. In a preferred embodiment, NK cells infusions will be administered in two week intervals.

For administration to patients, NK cells expanded according the disclosed methodologies can be administered in combination with (concomitantly or sequentially) a standard conditioning regimen. Such regimens are well established in the art and can include, e.g. cyclophosphamide and fludarabine. Without wishing to be bound by theory, conditioning regimens are thought to deplete host T cells and enable chimeric antigen receptor T (CAR-T) cells to expand, as well as enable the infused cells to utilize host cytokines, such as IL-15 and IL-7, fully. Such regimens are necessary for the survival of adoptively infused allogeneic NK cells, and are used even for autologous cell transfers.

When NK cells expanded according to the disclosed methods are administered in combination with cytokine support, exemplary cytokines for administration include IL-2, IL-15, ALT-803, hetIL-15, IL-12, IL-18, IL-21 or active fragments or derivatives thereof. Cytokine support is typically administered via s.c. or i.v. injection at doses ranging from 0.1 to 1000 μg/kg, preferably at doses of 10 μg/kg. Cytokine support can be co-administered, administered sequentially or administered concomitantly. In some aspects of the present disclosure, cytokine support can be administered up to two weeks, one week, or one day prior to NK cell infusion, or on the day of infusion. In some aspects of the present disclosure, administration of cytokine support can continue beyond the administration of NK cells. In a preferred aspect of the present disclosure, cytokine support will be administered weekly throughout the NK infusion cycle.

In addition to the need to obtain large quantities of highly active NK cells ex vivo, long term clinical development also requires strategies to maintain their activity in vivo. It has been shown that TGFβ significantly impairs NK function, thus, markedly impairs the in vivo activity of adoptively transferred NK cells (See e.g. Otegbeye, E. et al. Inhibiting TGF-beta signaling preserves the function of highly activated, in vitro expanded natural killer cells in AML and colon cancer models. PLOS One, 2018; 12(1):e019138. E pub 2018/01/18. Doi: 10.1371/journal.pone.0191358. Pub Med PMID: 29342200; PMCTD: PMC5771627.) There is also an established relationship between GSK3 activity and NK function. More specifically, GSK3-inhibited NK cells show significant efficacy in AML mouse models compared to non-GSK3-inhibited NK cells. (See e.g. Parameswaran P. et al. Suppression of GSK3 restores NK cell cytotoxicity in AML patients. Nature Communications. 2016; 7:1 1154. Doi 10.1038/ncommsl 1154. PubMed MPID: 27040177; PMCID: PMC4822012.) Thus, in some aspects of the disclosed methods, NK cells expanded according to the disclosed methods comprise co-administration, concomitant administration or sequential administration of NK cells and TGFβ and/or GSK3 inhibitors. In a preferred aspect of the present disclosure, treatment comprises co-administration of a TGFβ inhibitor, e.g. galunisertib, EW7203, EW7197, belagepumatucel-L, fresolimumab, gemogenovatucel-T, trabedersen, XOMA089. In a preferred aspect, the TGFβ inhibitor is galunisertib. TGF-beta and/or GSK3 inhibitors can be administered in doses ranging from 0.1 to 1000 μg/kg, and can be administered daily, weekly, monthly or dose frequency can be adjusted based on the needs of the patient.

Alternatively, TGF-beta signaling can be inhibited genetically, that is, by the genetic modification of the NK cells to express one or more proteins that impair TGF-beta signaling. This strategy has been successfully employed, for example, utilizing a dominant negative TGF-beta receptor II (TGF-beta RII), a gene encoding a truncated form of the transmembrane protein transforming growth factor, beta receptor II. (Chen, R. H. et al. Inactivation of the type II receptor reveals two receptor pathways for the diverse TGF-beta activities. Science 1993; 260 (5112): 1335-1338. Kloss, C. et al TGFBeta Signaling Blockade within PSMA Targeted CAR Human T Cells for the Eradication of Metastatic Prostate Cancer. Molecular Therapy 2016; 24(S1): S252-253.) In certain aspects of the disclosed methods, TGFβ signaling is impaired through the overexpression of the dominant negative TGF-beta RII, or by disrupting other TGFβ signaling molecules.

### EXAMPLES

### Development and Optimization of IL-21-Based Feeder Expansion Platform

The basis for the methodology employed herein is a genetically manipulated feeder cell line (so-called “NKF” cells) that comprises a leukemia cell line in which mbIL-21 has been expressed. In developing the NK cell expansion platform, the feeder cell line was selected to comprise cells that are efficiently lysed by NK cells and further exhibit a low level of expression of HLA class I molecules. A low level of HLA class I proteins is believed to be beneficial as certain epitopes are recognized by NK cell inhibitory killer inhibitory receptors (KIRs), which impair NK cell activation in vivo.

According to the disclosed methods, NKF cells are irradiated to prevent their expansion and then co-cultured with NK cells. NKF cells are rapidly killed by NK cells during the co-culture, leading to concomitant expansion of the NK cells. Unlike traditional NK cell expansion methods that are limited by rapid NK cell senescence and thus, limited proliferation, NK cells expanded by the disclosed process proliferated for extended durations (at least two months) and lead to robust proliferation (up to 30,000-fold within 4 to 5 weeks). Without wishing to be bound by theory, the prevention of senescence is believed to be due partially to the mbIL-21 on the feeder cells.

The novel NK cell expansion platform according to the present disclosure has been developed employing either OCI-AML3 or HL-60 cells, both of which are myeloid leukemia cells lines with the ability to expand rapidly, maintain good viability at high densities, and have low levels of HLA class I expression.

### Example 1—Development of NKF Cell Platform

To assess their expansion potential, OCI-AML3 (this line is commercially available from, e.g. DSMZ, Braunschweig, Germany, it is not available at ATCC) and HL-60 cells (also commercially available from, e.g., DSMZ, Braunschweig, Germany) cells were cultured in RPMI 1640 media (Hyclone) supplemented with 10% calf serum (Hyclone), penicillin (100 U/mL), and streptomycin (100 μg/mL). Testing for mycoplasma contamination was performed on all cell lines at regular intervals using the Mycoplasma Detection Kit-Quick Test (www.bimake.com). Once cultured and collected, the OCI-AML3 or HL-60 cells were irradiated using gamma irradiation (30 Gy) to obtain irradiated feeder cells.

Peripheral blood mononuclear cells (PBMC's) were isolated from peripheral blood of healthy human donors via FICOLL (GE Healthcare) gradient centrifugation. NK cells were isolated from PBMC's through magnetic bead CD3 depletion and in some cases, further purification was performed by CD56 isolation (Miltenyi Biotec). NK cells were cultured in RPMI 1640 media (Hyclone) supplemented with 10% calf serum (Hyclone), penicillin (100 U/mL), and streptomycin (100 μg/mL). To the culture media were added irradiated feeder cells (5:1 or 10:1 feeder cell:NK cell ratio, based on NK cell count on the day of addition) as well as IL-2 (range of 10-1000 units/mL) and the cells were incubated for 1 week at 37° C. in a tissue culture incubator with 5% CO₂. Referring to FIG. 2, the OCI-AML3 cell line (unfilled bar) led to a 4.6-fold expansion over a 1-week incubation period as measured by cell counting using a hematocytomer. The HL-60 cells (solid bar) led to a 2.9-fold expansion over this time period. Thus, OCI-AML3 cells demonstrated improved expansion capability compared to HL-60 cells.

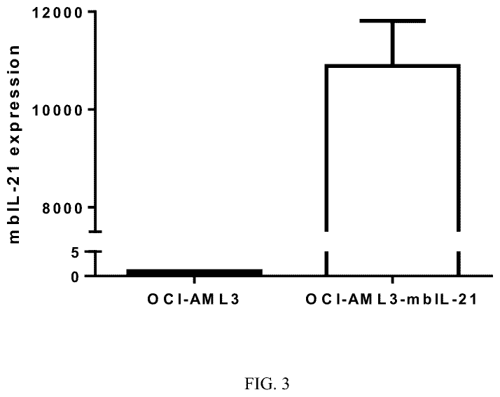

In order to improve on this expansion, OCI-AML3 cells were transduced to express membrane-bound IL-21. 293T cells were co-transfected with a lentiviral plasmid encoding mbIL-21 (VectorBuilder, Santa Clara, CA) and packaging plasmids VSVG and R8.74 using Turbofect (Thermo Fisher, Pittsburgh, PA). The polynucleotide sequence for the lentivirus plasmid for mbIL-21 expression is indicated in SEQ ID NO: 5 of the included sequence listing. Alternatively, 293T cells can be co-transfected with a lentiviral plasmid encoding mbIL-15 (SEQ ID NO: 6) or both mbIL-21 and mbIL-15 (SEQ ID NO:7). The studies shown herein employ cells expressing mbIL-21 only. Approximately 1 million OCI-AML3 cells were infected with 100 microliters of virus containing supernatant concentrated ˜20 fold overnight in 12.5% PEG in the presence of 6 μg/mL of polybrene. The virus was washed off the following day and stable cells lines were generated by selection with puromycin (1 μg/mL) approximately 72 hours after transduction. FIG. 3 reports an rt-PCR assessment of mbIL-21 expression in OCI-AML3 cells prior to transduction (solid bar) compared to OCI-ALM3 cells following transduction (unfilled bar). After lentivirus transduction, mbIL-21 is expressed at levels approximately 8,000-12,000 greater than native OCI-AML3 cells (data normalized). In some aspects of the present disclosure, the NKF cells comprise OCI-AML3 cells expressing mbIL-21.

As shown schematically in FIG. 1, following transduction with mbIL-21, NK cells isolated according to the procedure detailed above were co-cultured with irradiated NKF cells procured as detailed herein. To the NK-NKF cell co-culture was added IL-2. NKF cells were replenished weekly throughout the incubation period. Fresh IL-2 was added every 2-3 days during the co-culture period.

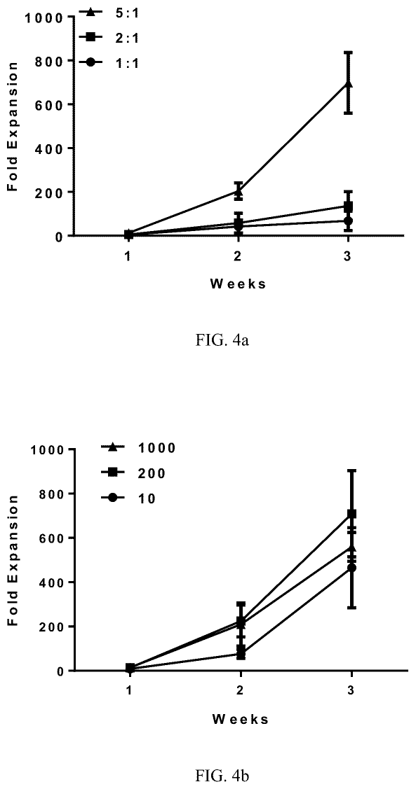

The herein described NKF expansion platform was optimized based on NKF-to-NK cell ratio in order to generate robust expansion while limiting the feeder cell number to avoid excess dead feeder cells in the final cell product. Using the cell culture procedure detailed above, NK cells were incubated for a period of 3 weeks, with once-weekly additions of NKF cells. The NKF cells were provided in ratios of 5:1, 2:1 and 1:1 NKF cells:NK cells based on a count of NK cells on the day of addition. Referring to FIG. 4a, after 3 weeks of expansion with NKF cells, the expansion platform comprising the step of adding NKF cells to the NK cell culture in a 5:1 ratio NKF:NK cells (based on cell count the day of the addition) resulted in an 8.3-fold higher expanded NK cell yield compared to a 1:1 ratio, and a 2.7-fold higher expanded NK cell yield compared to a 2:1 ratio. Further studies showed that even higher ratios (e.g. 10:1) led to even higher proliferation rates.

The NKF expansion platform was also optimized based on IL-2 concentration. Referring to FIG. 3b, NK cell expansion yields appeared to be independent of IL-2 concentration, with IL-2 concentrations of 10 U/mL (circles), 200 U/mL (squares) and 1000 U/mL (triangles) not resulting in a statistically significant difference in the proliferative capacity of the feeder cell expansion. Thus, in some aspects of the disclosed methods, the concentration of IL-2 employed the expansion platform ranges from about 10 u/mL to about 1000 U/mL, such as about 10 U/mL, about 50 U/mL, about 100 U/mL, about 150 U/mL, about 200 U/mL, about 250 U/mL, about 300 U/mL, about 350 U/mL, about 400 U/mL, about 450 U/mL, about 500 U/mL, about 550 U/mL, about 600 U/mL, about 650 U/mL, about 700 U/mL, about 750 U/mL, about 800 U/mL, about 850 U/mL, about 900 U/mL, about 950 U/mL, or about 1000 U/mL. In a particularly preferred aspect of the disclosed methods, the IL-2 concentration is 200 U/mL

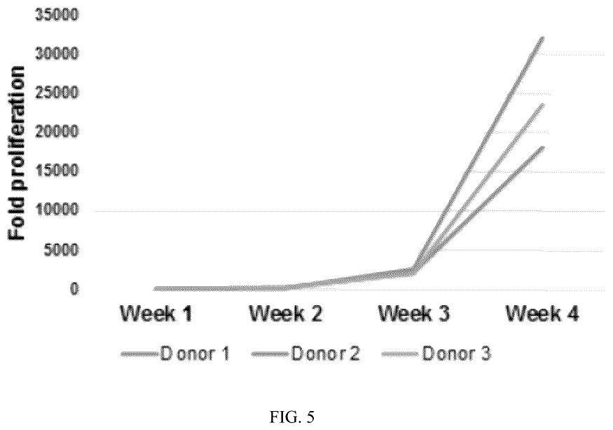

FIG. 5 demonstrates the robust proliferation of NKF cells produced according the present methods. NK cells were isolated from three healthy donors and expanded in the presence of irradiated NKF cells obtained as described above. NKF cells were added weekly in a 5:1 ratio (NKF:NK cells, based on NK cell count the day of the addition) for four weeks in media containing 200 U/mL IL-2. Expansion was rapid between weeks three and four, going from an expansion of about 2500-fold at three weeks to over 15,000-fold at the four week mark. At week four, the total NK cell expansion was between 18,000 and 32,000-fold.

### Example 2—Purity of NKF-Expanded NK Cells

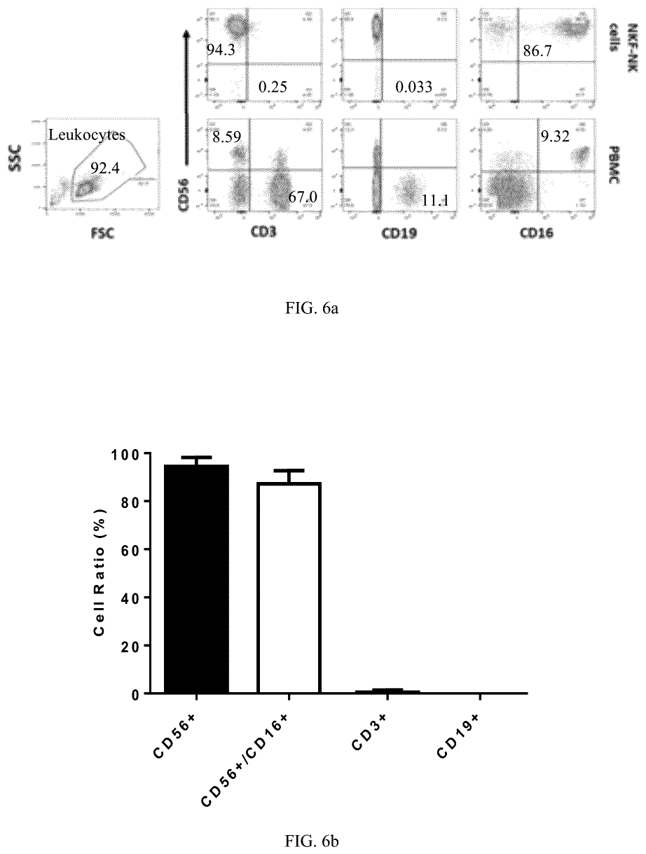

The purity of expanded NK cells was assessed by flow cytometry. FIG. 6a shows representative flow plots depicting the purity of NK cells in the initial PBMC starting population (bottom row) and after expansion with NKF cells (top row). After 2 weeks of expansion employing the methodology detailed herein, CD56+/CD3− cells were approximately 94% of the expanded cells, compared to less than 9% in the PBMC starting population (left column). CD3+ cells made up less than 1% in the expanded NK cells, compared to approximately two-thirds in the starting population (left column, top and bottom panels, respectively). B cells were virtually undetectable in the expanded NK cells which is important to avoid passenger B cells in the NK cell product. Approximately 87% of the expanded NK cells were CD56+/CD16+(FIG. 6a, right column, top panel), suggesting that a large portion of these cells could mediate antibody-dependent cell-mediated toxicity (ADCC). This is in contrast to only about 9% of the NK cells in the pre-processed PBMC fraction were CD56+/CD16+(FIG. 6a, right column, bottom panel). Low T cell contamination post-expansion is important for avoiding potential GVHD after expanded NK cells are utilized as universal donor cells for treating cancer patients, irrespective of HLA matching.

### Example 3—Cytotoxic Activity of NKF-Expanded NK Cells

Traditionally, NK cells utilized for adoptive cell therapy are activated by high dose IL-2 prior to administration to patients. It is well established that IL-2 improves cellular cytotoxicity, although the mechanisms for this are not well understood. Without wishing to be bound by theory, it has been hypothesized that the presence of mb IL-21 can prevent NK cells from undergoing senescence, markedly improving their ability to expand ex vivo.

While this IL-2 treatment leads to a relatively high level of NK cell cytotoxic activity, NK cells expanded with the NKF platform disclosed herein unexpectedly exhibited significantly higher activity than NK cells activated with IL-2 according to known methods. NK cell cytotoxic function was assessed by measuring the number of live cells identified by calcein-AM (CAM) labeling. Target cells and NK cells were labeled with CAM (BD Pharmingen, San Jose, CA) and calcein-violet (CV) (eBioscience from ThermoFisher), respectively for approximately 15 minutes in the dark at room temperature. NK cells were co-incubated with target cells at the indicated ratios for 4 hours in triplicate at 37° C. in a tissue culture incubator with 5% CO₂, and the samples were analyzed by flow cytometry (Attune NXT, Invitrogen) in 96 well plates. The CV-positive NK cells were gated out for analysis. Percent lysis was calculated according to the equation below:

$$\frac{\begin{matrix}( \# CAM bright target cells alone ) - \\ ( \# CAM bright cells in NK and target co - culture )\end{matrix}}{\# CAM bright target cells alone} \times 100$$

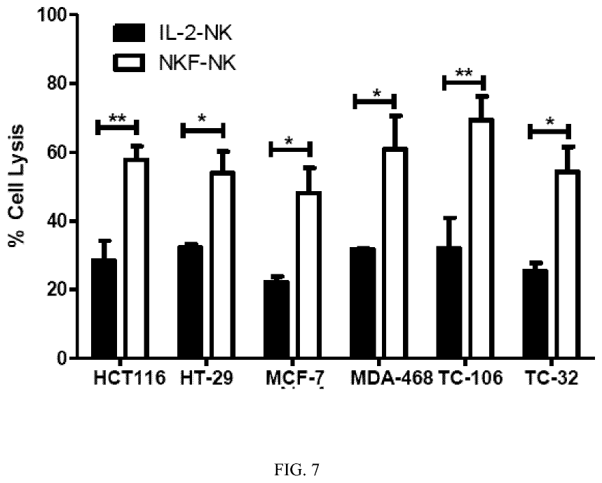

FIG. 7 shows the results of cytotoxic activities studies for a variety of cancer cells lines. NK cells expanded with NKF cells or freshly isolated NK cells activated with IL-2 (200 U/mL) overnight were incubated with the indicated cancer cells (from left to right in FIG. 7, x-axis: HCT116, HT-29, MCF-7, MDA-468, TC-106 and TC-32) which had been prelabeled with Calcein AM. The ratio of NK cells:target (cancer) cells was 1:1. After 4 hours, the above-described flow cytometry-based cytotoxicity assay was performed. In all cases, NK cells expanded with NKF cells (open bars) demonstrated significantly greater cytotoxic activity compared to freshly isolated NK cells activated with IL-2 (solid bars). The increase in cell lysis (FIG. 7, y-axis) for the NK cells expanded by the NKF platform (FIG. 5, open bars) varied across the cancer cell lines tested from about a 30% increase to an increase of over 50% compared to non-expanded NK cells activated overnight with IL-2 treatment (FIG. 5, solid bars).

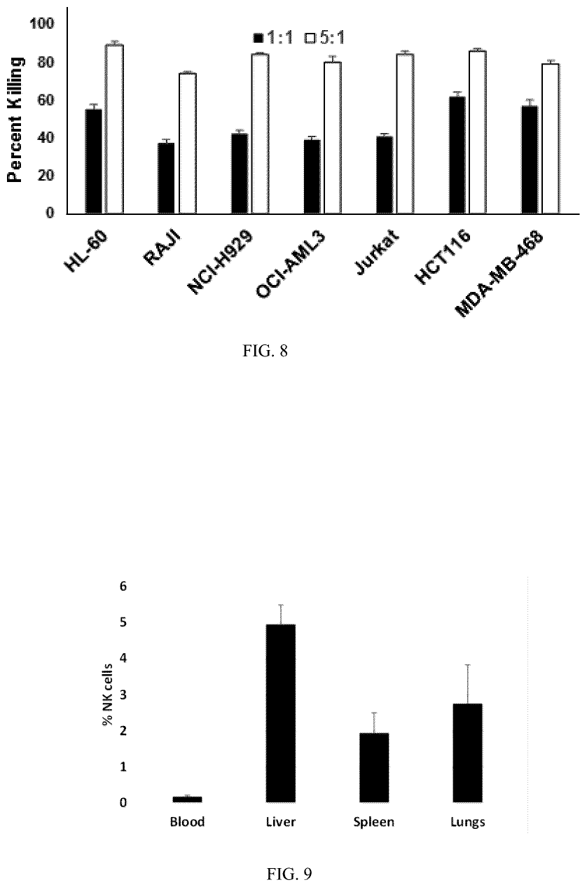

Further, high levels of in vitro cytotoxic activity utilizing the NKF expansion platform were observed across a variety of cancer cell types, including colon, sarcoma, breast cancer and multiple blood cancer cell types. Referring to FIG. 8, NKF-expanded NK cells were tested in a 1:1 (solid bars) and a 5:1 (open bars) NK cell:target cell ratio using the same flow cytometry assay employed for the cell lysis assay described immediately above. As demonstrated in FIG. 8, even at low NK:target cell ratios (1:1), NKF-expanded NF cells demonstrate excellent sensitivity to a variety of cancer cells (e.g., from left to right in FIG. 8: HL-60, RAJI, NCI-H929, OCI-AML3, Jurkat, HCT116, and MDA-MB-468). For example, when employed in a 1:1 NK:target cell ratio, the percent killing (FIG. 8, y-axis) ranged from about 35% to about 65%. When the NK:target ratio was increased to 5:1, the percent killing increased from about 75% to about 90%.

Without wishing to be bound by theory, the increased cell killing observed when using the NKF expansion platform reported herein appears to be due to a significant increase in NK cell activating receptors (e.g. NKG2D and NKp30/NKp44), as well as an increase in cell trafficking molecules such as CXCR6 for the in vivo studies. NKp30 and NKp44 are known to play key roles in NK cell killing of colon cancer cells, and CXCR6 is involved in the trafficking of NK cells to the liver, the most common site of colon cancer metastases. In addition to the liver, the NKF-expanded NK cells show a high propensity to traffic to the lungs, making these ideal targets for immunotherapy treatment. Experimentally, NKF-expanded NK cells were labelled with CFSE and injected i.v. into NSG mice. After a 72 hours, single cell suspensions from the indicated organs were prepared and assessed by flow cytometry. As demonstrated in FIG. 9, the organs that were the highest targeted by the NKF-expanded NK cells were the liver (5%) and lungs (3%), followed by the spleen (2%). Very few NK cells were observed in the blood (>0.3%) under these conditions.

### Enhancement of Cytotoxic Activity

Not only are large numbers of NK cells necessary for infusion into patients to achieve clinical efficacy, but these cells must also survive and proliferate in vivo for a sustained duration. After infusion of NK cells into patients, cytokine support is used to enable the cells to survive and proliferate. While low dose IL-2 has traditionally been used, it can lead to unwanted proliferation of regulatory T cells and suboptimal NK cell proliferation. IL-15 support has demonstrated better results, however, its use is limited as it requires extremely high doses for meaningful effects and it has an extremely short half-life. As noted above, ALT-803 has shown therapeutic promise in multiple cancer models, but had not been tested in combination with adoptively transferred NK cells.

ALT-803 was tested to assess its potential as cytokine support for adoptive NK therapy in cancer mouse models.

### Example 3a—In Vitro Assessment

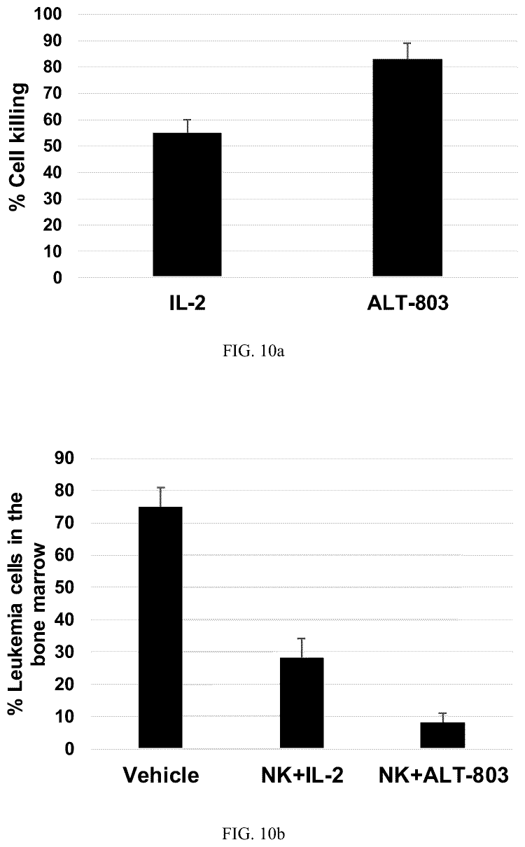

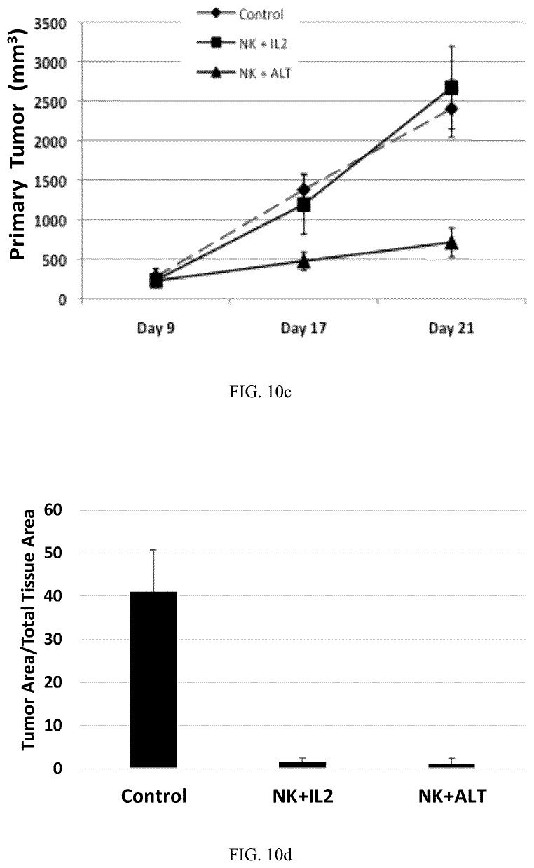

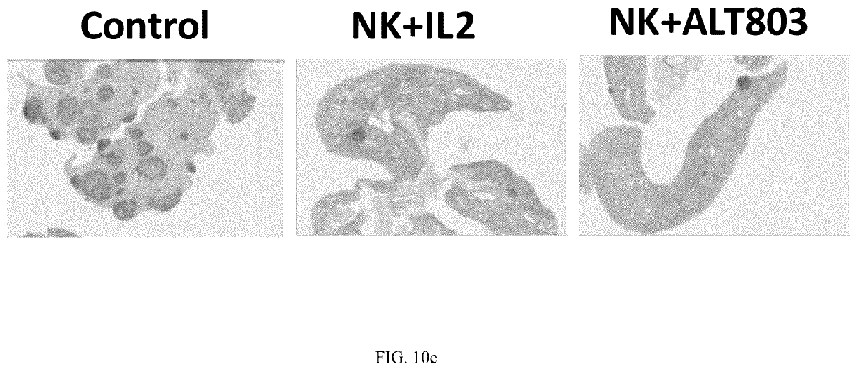

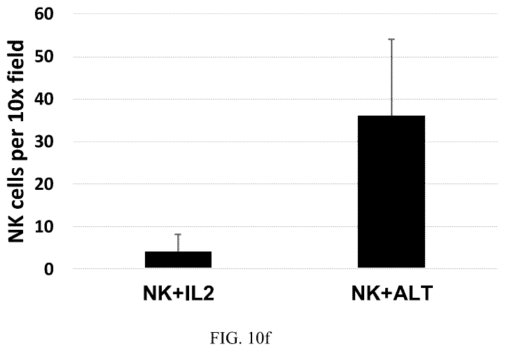

The activity of NKF-expanded NK cells was tested in combination with IL-2 and ALT-803 according to the cytometry assay described above. NK cells were treated with IL-2 (200 units/mL) or ALT-803 (100 ng/mL) and cytotoxicity against OCI-AML3 cells was measured in vitro using the Calcein AM assay described above. As demonstrated in FIG. 10, both IL2- and ALT-803-supported NK cells resulted in high levels of cytotoxicity. IL-2-supported cells resulted in about 50-60% cytotoxicity (FIG. 10a, left bar). ALT-803-supported cells resulted in about 70-90% killing efficacy (FIG. 10a, right bar). Thus, while both provided functional cytokine support, the activity of NKF-expanded NK+ALT-803 was enhanced by about 33% compared to the activity of NKF-expanded NK+IL-2 in in vitro models.

### Example 3b—Mouse Cancer Models

Employing an AML mouse model, NSG mice (n=5 per group) were injected i.v. with OCI-AML3 cells, and five days later, the mice were injected with 10×10⁶ NKF-expanded NK cells or with vehicle i.v. weekly for 2 weeks. Mice receiving NK cells also received IL-2 or ALT-803 cytokine support. After 4 weeks, the mice were sacrificed and bone marrow was assessed for leukemic burden (FIG. 10b) by flow cytometry as described above. Referring to FIG. 10b, the NK cell treatment led to a significant reduction in leukemia burden for both groups compared to the control vehicle (FIG. 10b, left bar). Mice administered only the control vehicle demonstrated a leukemic burden of about 70-80%. In contrast, mice administered NK cell treatment concomitantly with IL-2 cytokine support demonstrated a leukemic burden of about 25-35% (FIG. 10b, center bar). Mice administered NK cell treatment in combination with ALT-803 cytokine support (FIG. 10b, right bar) had a leukemic burden of about 8-12%. Thus, while treatment with NK cells and cytokine support was significantly improved over vehicle alone, ALT-803 cytokine support was more than twice as effective as IL-2 cytokine support.

The NKF-expanded NK cells were also tested in a lung metastases model of human sarcoma using TC-106 Ewing's sarcoma cells. In this experiment, 1×10⁵ TC-106 cells were injected subcutaneously into the flanks of NSG mice (n=5 per group). Five million NKF-expanded NK cells were injected i.v. weekly for 2 weeks once tumors reached approximately 100 mm³. The mice were sacrificed after approximately 4 weeks and tumor volume was quantified using standard techniques and the VENTANA imaging software (Ventana Medical Systems, ROCHE Group). Referring to FIG. 10c, mice treated with NK+ALT-803 (triangles, solid line) demonstrated a significant reduction in primary tumor volume compared to NK+IL2 treatment (squares, solid line) or to a vehicle control (PBS) (diamonds, dashed line). More specifically, at Day 21, mice treated with NK cells employing ALT-803 as cytokine support were found to have primary tumors from about 500 mm³ to about 900 mm³. In contrast, there was no statistical difference in tumor size for NK cell+IL-2 support and control only, both of which resulted in tumors over 2000 mm³ in volume.

Referring to FIGS. 10d and 10e, NKF-expanded NK cell treatment (both ALT-803 and IL-2 treated groups) had dramatic reductions in lung metastases as seen by Ki67 stained tissue sections. FIG. 10d reports nearly a 40-fold reduction in lung metastases (based on the ratio of tumor area:total tissue area, y-axis) for NK cell-treatment regardless of cytokine support, as compared to control. FIG. 10e shows exemplary staining of control (left panel), NK+IL2 (center panel) and NK+ALT-803 (right panel) lung tumor metastases.

Human specific CD45 antibodies were used to monitor NK cell trafficking. Dissected lungs from the mice was fixed in formalin overnight and then paraffin blocks were prepared. The tissues were cut and slides were prepared from the paraffin blocks. Immunohistochemistry was performed by staining the slides with a human specific CD45 antibody to identify the human NK cells. Quantification of NK cell number (based on counting four 10× fields of lung tissue sections stained with CD45 antibodies) is presented in FIG. 10f and demonstrates that in mice treated with NK cells in combination with ATL-803 as cytokine support, significantly higher levels of NK cells were present in the lung (right bar) compared to NK-IL cells for mice treated with NK cells and IL-2 support (left bar). This suggests that ALT-803 demonstrates improved trafficking to the tumor site as compared to IL-2.

TGF-beta inhibition improves the efficacy of NK cells in a metastatic colon cancer model.

While NK cells alone demonstrate efficacy in mouse models, it is known that tumor microenvironment factors impair NK cell function and limit their in vivo efficacy. One major immunoregulatory factor that regulates NK cell function is TGF-beta. Not only does TGF-beta markedly impair NK cell killing of cancer cells, but it can be readily reversed by TGF-beta inhibitors such as galunisertib.

### Example 4—Effect of TGF-Beta Inhibition on Cytotoxic Activity

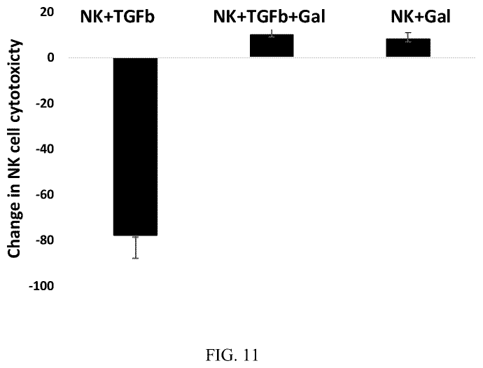

Referring to FIG. 11, expanded NK cells (K562 cells expressing mbIL-21) were treated with TGF-beta (5 ng/mL), galunisertib, or a combination thereof for 72 hours. The calcein-AM label flow cytometry assay described above was used against OCI-AML3 target cells at a 5:1 NK:target cell ratio to demonstrate that when NK cells are co-administered with TGF-beta, there is a drop in killing efficacy ranging from about 70% to about 90% (left bar). That drop can be rescued and reversed by co-administering NK cells with galunisertib (middle and right bars), in which case killing efficacy was improved by about 10%.

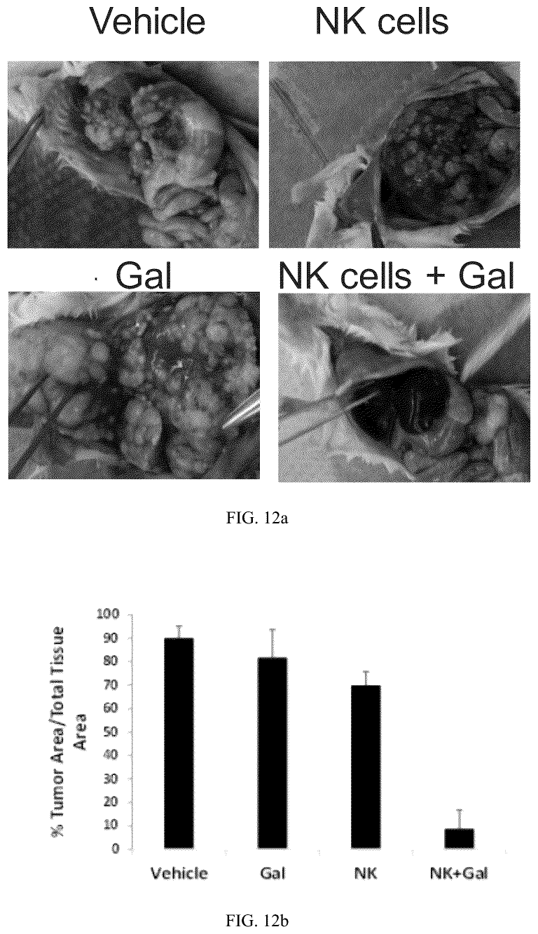

Inhibition of TGF-beta also dramatically improves the in vivo activity of adoptively infused NK cells in a metastatic colon cancer model. NSG mice (n=5 per group) were injected into the spleen with HCT116 cells to initiate colon metastases to the liver followed by hemisplenectomy. The mice were treated with NK cells i.v., galunisertib (75 mg/kg daily PO) or both for 3 weeks. The mice were sacrificed after 4 weeks. Quantification of tumor in sections from the liver (n=4 per group) using Ventana Image Viewer. Referring to FIG. 12a, representative gross images are shown of liver tumor section following treatment with vehicle (top left panel), NK cells alone (top right panel), galunisertib alone (bottom left panel) and NK cells with concomitant administration of galunisertib (bottom right panel). These results are quantified in FIG. 12b as a ratio of % tumor area:total tissue are. Whereas the liver tumor burden of the control mice was about 90%, the concomitant administration of NK cells and galunisertib was about 10%.

What is claimed is:

1.-60. (canceled)

61. A method of treating a recipient subject in need thereof comprising administering ex vivo expanded NK cells, wherein the expanded NK cells are obtained by
(a) harvesting natural killer (NK) cells from an NK cell donor to obtain donor NK cells;
(b) isolating the donor NK cells; and
(c) expanding the donor NK cells in the presence of acute myeloid leukemia (AML) feeder cells engineered to express a membrane-bound interleukin (mbIL) protein comprising a polypeptide having at least 98% sequence homology with SEQ ID NO:1 to obtain expanded NK cells.

62. The method of claim 61, wherein the membrane-bound interleukin (mbIL) protein comprising a polypeptide has at least 99% sequence homology with SEQ ID NO:1.

63. The method of claim 62, wherein the membrane-bound interleukin (mbIL) protein consists of a polypeptide of SEQ ID NO:1.

64. The method according to claim 61, wherein the donor NK cells are harvested from a living donor.

65. The method according to claim 64, wherein the donor NK cells are harvested from the recipient subject in need thereof.

66. The method according to claim 64, wherein the donor NK cells are allogenic.

67. The method according to claim 61, wherein the donor NK cells are harvested from cord blood.

68. The method according to claim 61, wherein the expanded NK cells are administered concomitantly or sequentially with at least one additional therapeutic.

69. The method according to claim 68, wherein the at least one additional therapeutic is selected from the group consisting of a cytokine support, an inhibitor of an immunoregulatory protein, or a combination thereof.

70. The method according to claim 69, wherein the expanded NK cells are administered in combination with a cytokine support.

71. The method according to claim 70, wherein the cytokine support is selected from the group consisting of IL-2, IL-15, ALT-803, hetIL-15, IL-12, IL-18, IL-21 and active derivatives or active fragments thereof.

72. The method according to claim 70, wherein the cytokine support is administered sequentially or administered concomitantly.

73. The method according to claim 70, wherein the cytokine support is administered no more than two weeks prior to administration of the expanded NK cells.

74. The method according to claim 70, wherein the cytokine support is administered weekly while treating the recipient subject in need thereof.

75. The method according to claim 70, wherein the cytokine support is administered in a dose ranging from 0.1 to 1000 μg/kg.

76. The method according to claim 69, wherein the at least one additional therapeutic is an inhibitor of an immunoregulatory protein selected from the group consisting of a TGFβ inhibitor, GSK3 inhibitor, PD1 inhibitor, PDL1 inhibitor, a LAG-3 inhibitor, a TIM-3 inhibitor, a TIGIT inhibitor, derivatives thereof, and combinations thereof.

77. The method according to claim 61, wherein the recipient subject in need thereof has a cancer or a viral infection.

78. The method according to claim 77, wherein the subject in need thereof has a cancer that is a solid cancer selected from the group consisting of colon cancer, a prostate cancer, a sarcoma, a pancreatic cancer, and a breast cancer.

79. The method according to claim 77, wherein the subject in need thereof has a cancer that is a blood cancer selected from the group consisting of acute myeloid leukemia (AML), myeloma, T cell leukemia, Non-Hodgkin's Lymphoma and B cell leukemia, Myelodysplastic syndromes, chronic lymphocytic leukemia (CLL), and chronic myeloid leukemia (CML).

80. The method according to claim 77, wherein the subject in need thereof has a viral infection selected from the group consisting of coxsackievirus, human immunodeficiency virus (HIV), hepatitis C virus (HCV), influenza virus, poxvirus, and herpesvirus.

81. The method according to claim 61, wherein the expanded NK cells are administered in a dose of at least 10⁶ cells/kg.

82. The method according to claim 81, wherein the expanded NK cells are administered in a dose of about 1,000×10⁶ cells/kg.

83. The method according to claim 61, wherein the expanded NK cells are administered to the recipient subject in need thereof in two-week intervals.

84. The method according to claim 83, wherein the expanded NK cells are administered in combination with a cytokine support, wherein the cytokine support is administered weekly.

85. The method according to claim 84, wherein the recipient subject in need thereof has a blood cancer.


SEQUENCE LISTING (7 sequences)

SEQ ID NO: 1 - protein, 395 aa, synthetic construct
  REGION 1..395: note = Synthetic construct mbIL-21
MLLLVTSLLLCELPHPAFLLIPQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFIESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFF

SEQ ID NO: 2 - DNA, 1185 bp, synthetic construct
  misc_feature 1..1185: note = Synthetic construct mbIL-21
  source 1..1185: mol_type = other DNA
atgctcttgctcgtcacctccttgctgctgtgtgaactgccgcaccctgcgttccttcttattccacaggaccgacacatgatccgaatgcgccaactcatagacatcgtagaccagctcaagaattatgtcaacgacctggtgcctgagttcctcccggctccagaggacgtcgaaacaaactgcgaatggtctgcttttagctgttttcaaaaggcgcagttgaaatcagcgaacacgggcaataatgagcgcattattaacgtctcaatcaagaaactgaaaagaaagccaccgtcaacaaatgctgggcgacggcagaaacatagacttacctgcccaagctgtgactcctacgagaaaaagccgccgaaagagtttcttgaaaggttcaagagcctcctccagaaggtgtctaccctgagcttcatcgaatccaagtacggtcccccatgtcccccgtgccccgcccctgagtttctggggggtccttcagtttttctgttcccgccaaagccaaaggacactctgatgatatccagaacaccggaggttacttgtgtcgtggtcgatgttagccaagaggaccccgaagttcagttcaactggtatgtcgacggcgtggaagtccataatgcaaaaacgaaaccacgggaagagcaattcaatagcacctacagagtagtatcagttctgaccgttctgcatcaagactggctcaacggtaaagaatacaagtgcaaagtgtctaataaaggcttgcctagttccatagaaaagaccatcagcaaggcgaaagggcaaccacgagagccacaagtctatacgcttcccccatcacaagaagaaatgacgaagaaccaagtgagcttgacgtgcttggtgaagggtttttacccgtcagacatagccgtcgagtgggaatcaaatggccagcccgagaacaattacaaaacaactccacccgttctcgactccgacggttcctttttcctctactcacgattgactgtagataagagcagatggcaagagggaaatgtgttctcttgtagcgtgatgcacgaagcactccataaccactacactcagaaatcactctctctcagtcttgggaaaatggcactcattgtgctcggtggggtggcaggtcttcttctgtttattggccttggaatatttttc

SEQ ID NO: 3 - protein, 387 aa, synthetic construct
  REGION 1..387: note = Synthetic construct mbIL-15
MLLLVTSLLLCELPHPAFLLIPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFF

SEQ ID NO: 4 - DNA, 1164 bp, synthetic construct
  misc_feature 1..1164: note = Synthetic construct mbIL-15
  source 1..1164: mol_type = other DNA
atgctgcttctcgtaacgtccttgttgttgtgcgaattgcctcatccagcattcttgcttattcccaactgggtcaacgtaataagtgacttgaaaaaaatcgaggatctgatacagagtatgcacatcgatgcaacactctacactgagagtgatgttcacccgagttgtaaagtgacagcgatgaaatgttttcttcttgaacttcaggttatatcacttgaatcaggtgatgctagtatccatgacacggtggagaatctcatcattcttgctaataacagtctctcctcaaatggtaatgtaacagaatctggctgtaaagaatgcgaggaattggaggagaagaacataaaggaatttctccagagcttcgtccatatagtacagatgttcatcaacacatccgagtctaaatacggcccgccgtgtccaccgtgtccagcgccagagtttctcggtggcccttctgtgttcctgttcccccccaagcccaaagatacccttatgatatcaagaacaccagaagtcacttgtgtcgttgtcgatgtgagccaagaagaccctgaggtacagttcaactggtatgtagatggggttgaggttcataacgccaaaactaagcccagagaagaacaattcaatagtacttatagagttgtctcagtacttacggtcttgcatcaagattggttgaatggtaaggaatataagtgcaaagtgtccaataagggcctgccaagcagtatagagaagactatctccaaggcaaaagggcaaccccgcgaaccacaggtctatactcttccacccagccaagaggaaatgactaaaaatcaagtatctcttacgtgccttgtgaaaggtttttacccgagtgatattgccgtagagtgggaatccaacggccagccagagaacaactataagactacgccgccggttttggatagtgacggctccttctttctgtactccaggctcacggtcgacaagagtcgatggcaagaggggaacgtatttagttgctctgtgatgcatgaagcgctgcacaatcattatactcagaagagtctgtccctgagtctcggcaaaatggccctgattgtactgggaggggttgctggacttctcctgtttatcggactcggtatcttcttctga

SEQ ID NO: 5 - DNA, 9680 bp, synthetic construct
  misc_feature 1..9680: note = Synthetic construct
  source 1..9680: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatgctcttgctcgtcacctccttgctgctgtgtgaactgccgcaccctgcgttccttcttattccacaggaccgacacatgatccgaatgcgccaactcatagacatcgtagaccagctcaagaattatgtcaacgacctggtgcctgagttcctcccggctccagaggacgtcgaaacaaactgcgaatggtctgcttttagctgttttcaaaaggcgcagttgaaatcagcgaacacgggcaataatgagcgcattattaacgtctcaatcaagaaactgaaaagaaagccaccgtcaacaaatgctgggcgacggcagaaacatagacttacctgcccaagctgtgactcctacgagaaaaagccgccgaaagagtttcttgaaaggttcaagagcctcctccagaaggtgtctaccctgagcttcatcgaatccaagtacggtcccccatgtcccccgtgccccgcccctgagtttctggggggtccttcagtttttctgttcccgccaaagccaaaggacactctgatgatatccagaacaccggaggttacttgtgtcgtggtcgatgttagccaagaggaccccgaagttcagttcaactggtatgtcgacggcgtggaagtccataatgcaaaaacgaaaccacgggaagagcaattcaatagcacctacagagtagtatcagttctgaccgttctgcatcaagactggctcaacggtaaagaatacaagtgcaaagtgtctaataaaggcttgcctagttccatagaaaagaccatcagcaaggcgaaagggcaaccacgagagccacaagtctatacgcttcccccatcacaagaagaaatgacgaagaaccaagtgagcttgacgtgcttggtgaagggtttttacccgtcagacatagccgtcgagtgggaatcaaatggccagcccgagaacaattacaaaacaactccacccgttctcgactccgacggttcctttttcctctactcacgattgactgtagataagagcagatggcaagagggaaatgtgttctcttgtagcgtgatgcacgaagcactccataaccactacactcagaaatcactctctctcagtcttgggaaaatggcactcattgtgctcggtggggtggcaggtcttcttctgtttattggccttggaatatttttcgcccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtggccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 6 - DNA, 9652 bp, synthetic construct
  misc_feature 1..9652: note = Synthetic construct
  source 1..9652: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggcatgctgcttctcgtaacgtccttgttgttgtgcgaattgcctcatccagcattcttgcttattcccaactgggtcaacgtaataagtgacttgaaaaaaatcgaggatctgatacagagtatgcacatcgatgcaacactctacactgagagtgatgttcacccgagttgtaaagtgacagcgatgaaatgttttcttcttgaacttcaggttatatcacttgaatcaggtgatgctagtatccatgacacggtggagaatctcatcattcttgctaataacagtctctcctcaaatggtaatgtaacagaatctggctgtaaagaatgcgaggaattggaggagaagaacataaaggaatttctccagagcttcgtccatatagtacagatgttcatcaacacatccgagtctaaatacggcccgccgtgtccaccgtgtccagcgccagagtttctcggtggcccttctgtgttcctgttcccccccaagcccaaagatacccttatgatatcaagaacaccagaagtcacttgtgtcgttgtcgatgtgagccaagaagaccctgaggtacagttcaactggtatgtagatggggttgaggttcataacgccaaaactaagcccagagaagaacaattcaatagtacttatagagttgtctcagtacttacggtcttgcatcaagattggttgaatggtaaggaatataagtgcaaagtgtccaataagggcctgccaagcagtatagagaagactatctccaaggcaaaagggcaaccccgcgaaccacaggtctatactcttccacccagccaagaggaaatgactaaaaatcaagtatctcttacgtgccttgtgaaaggtttttacccgagtgatattgccgtagagtgggaatccaacggccagccagagaacaactataagactacgccgccggttttggatagtgacggctccttctttctgtactccaggctcacggtcgacaagagtcgatggcaagaggggaacgtatttagttgctctgtgatgcatgaagcgctgcacaatcattatactcagaagagtctgtccctgagtctcggcaaaatggccctgattgtactgggaggggttgctggacttctcctgtttatcggactcggtatcttcttctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtggccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 7 - DNA, 10907 bp, synthetic construct
  misc_feature 1..10907: note = Synthetic construct
  source 1..10907: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatgctcttgctcgtcacctccttgctgctgtgtgaactgccgcaccctgcgttccttcttattccacaggaccgacacatgatccgaatgcgccaactcatagacatcgtagaccagctcaagaattatgtcaacgacctggtgcctgagttcctcccggctccagaggacgtcgaaacaaactgcgaatggtctgcttttagctgttttcaaaaggcgcagttgaaatcagcgaacacgggcaataatgagcgcattattaacgtctcaatcaagaaactgaaaagaaagccaccgtcaacaaatgctgggcgacggcagaaacatagacttacctgcccaagctgtgactcctacgagaaaaagccgccgaaagagtttcttgaaaggttcaagagcctcctccagaaggtgtctaccctgagcttcatcgaatccaagtacggtcccccatgtcccccgtgccccgcccctgagtttctggggggtccttcagtttttctgttcccgccaaagccaaaggacactctgatgatatccagaacaccggaggttacttgtgtcgtggtcgatgttagccaagaggaccccgaagttcagttcaactggtatgtcgacggcgtggaagtccataatgcaaaaacgaaaccacgggaagagcaattcaatagcacctacagagtagtatcagttctgaccgttctgcatcaagactggctcaacggtaaagaatacaagtgcaaagtgtctaataaaggcttgcctagttccatagaaaagaccatcagcaaggcgaaagggcaaccacgagagccacaagtctatacgcttcccccatcacaagaagaaatgacgaagaaccaagtgagcttgacgtgcttggtgaagggtttttacccgtcagacatagccgtcgagtgggaatcaaatggccagcccgagaacaattacaaaacaactccacccgttctcgactccgacggttcctttttcctctactcacgattgactgtagataagagcagatggcaagagggaaatgtgttctcttgtagcgtgatgcacgaagcactccataaccactacactcagaaatcactctctctcagtcttgggaaaatggcactcattgtgctcggtggggtggcaggtcttcttctgtttattggccttggaatatttttcggaagcggagagggcaggggaagtcttctaacatgcggggacgtggaggaaaatcccggccccatgctgcttctcgtaacgtccttgttgttgtgcgaattgcctcatccagcattcttgcttattcccaactgggtcaacgtaataagtgacttgaaaaaaatcgaggatctgatacagagtatgcacatcgatgcaacactctacactgagagtgatgttcacccgagttgtaaagtgacagcgatgaaatgttttcttcttgaacttcaggttatatcacttgaatcaggtgatgctagtatccatgacacggtggagaatctcatcattcttgctaataacagtctctcctcaaatggtaatgtaacagaatctggctgtaaagaatgcgaggaattggaggagaagaacataaaggaatttctccagagcttcgtccatatagtacagatgttcatcaacacatccgagtctaaatacggcccgccgtgtccaccgtgtccagcgccagagtttctcggtggcccttctgtgttcctgttcccccccaagcccaaagatacccttatgatatcaagaacaccagaagtcacttgtgtcgttgtcgatgtgagccaagaagaccctgaggtacagttcaactggtatgtagatggggttgaggttcataacgccaaaactaagcccagagaagaacaattcaatagtacttatagagttgtctcagtacttacggtcttgcatcaagattggttgaatggtaaggaatataagtgcaaagtgtccaataagggcctgccaagcagtatagagaagactatctccaaggcaaaagggcaaccccgcgaaccacaggtctatactcttccacccagccaagaggaaatgactaaaaatcaagtatctcttacgtgccttgtgaaaggtttttacccgagtgatattgccgtagagtgggaatccaacggccagccagagaacaactataagactacgccgccggttttggatagtgacggctccttctttctgtactccaggctcacggtcgacaagagtcgatggcaagaggggaacgtatttagttgctctgtgatgcatgaagcgctgcacaatcattatactcagaagagtctgtccctgagtctcggcaaaatggccctgattgtactgggaggggttgctggacttctcctgtttatcggactcggtatcttcttctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtggccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt